(19) United States
(12) Patent Application Publication
(10) Pub. No.: US 2015/0142080 A1
(43) Pub. Date: May 21, 2015

(54) TREATMENT OR THERAPEUTIC APPARATUS

(71) Applicant: WTL TECHNOLOGIES LIMITED, Coventry (GB)

(72) Inventor: Len Maxwell, Coventry (GB)

(21) Appl. No.: 14/401,794

(22) PCT Filed: May 17, 2013
(86) PCT No.: PCT/GB2013/000224

§ 371 Date: Nov. 17, 2014

(30) Foreign Application Priority Data
May 17, 2012 (GB) 1208727.6

(51) Int. Cl. A61N 1/36; A61N 1/04; A61N 1/372

(52) U.S. Cl. CPC A61N 1/36014; A61N 1/37235; A61N 1/37252; A61N 1/0484; A61N 1/0476
USPC 607/59

(57) ABSTRACT

Apparatus for treatment of an animal or human is disclosed which comprises a pulse delivery system for generating a series of electrical current pulses having a pre-programmed waveform. A flexible belt or belt-like carrier is provided and arranged in use to be secured to, on or around an animal or human and which comprises a pair of spaced-apart electrodes for making contact with the skin of the animal or human, and a connector for electrically connecting an output terminal of the pulse delivery system to the electrodes so that in use the current pulses are delivered between the electrodes across or through underlying tissue of the animal or human.

### FIELD OF THE INVENTION

This invention relates to an apparatus for the treatment or therapy of an animal or human, particularly an apparatus that passes current across or through tissue of the animal or human.

### BACKGROUND OF THE INVENTION

For many years so-called micro current technology has been used to treat various injuries or ailments. This generally involves passing pulses of electrical current between spaced-apart electrode pads placed on the wearer's skin.

Traditionally, these devices tended to be for human use only, bulky, limited in terms of their applications and effectiveness, and required the user to go to, and be treated by, a practitioner.

Traditionally, micro current delivery systems are designed and intended for single, or very limited clinical applications.

### SUMMARY OF THE INVENTION

According to one aspect of the invention, there is provided apparatus for treatment of an animal or human comprising: a pulse delivery system for generating a series of electrical current pulses having a pre-programmed waveform; and a flexible belt or belt-like carrier arranged in use to be secured to, on or around an animal or human and comprising a pair of spaced-apart electrodes for making contact with the skin of the animal or human, and a connector for electrically connecting an output terminal of the pulse delivery system to the electrodes so that in use the current pulses are delivered between the electrodes across or through underlying tissue of the animal or human.

The apparatus provides a convenient way of applying micro currents to the human or animal body in an easily-worn device and which can deliver a programmed waveform which can be set to treat a particular or range of problems and ailments using a selected sequence of so-called Programmes which define the characteristics of the current waveform delivered.

Research by the Applicant has indicated that the treatment delivered by the apparatus may be “systemic” in that, once introduced, the current circulates the entire body and will naturally gravitate to areas of imbalance or abnormality, both physical, chemical or electrical.

It is irrelevant, therefore as to where on a body that the apparatus is used (and therefore where the current is introduced.) It does not need to be local to the area of concern.

In the preferred embodiment, the apparatus is configured to deliver a selected sequence of Programmes, each Programme having different respective waveform characteristics.

The Programmes can be grouped into two types, each grouping having a waveform of similar shape. In a first group, the the Applicant has found that the waveforms, when applied, promote rebalancing, the reduction of swelling, bruising, pain, inflammation, hormonal imbalances, autoimmune conditions, psychological conditions and help boost the immune system. In a second group, the Applicant has found that the waveforms, when applied, effectively kick-start the body's regenerative healing function to repair damaged tissue with reduced or little scarring. Within each group, the Programmes have a similar waveform shape characteristics—in the first group the Programmes use alternative positive and negative polarity for bunches of pulses; in the second group, only positive polarity pulse bunches are used.

Also, within a given group, there are defined ON/OFF times with one Programme within the group having shorter ON/OFF times than the other Programme within the group; delivery of the shorter ON/OFF time Programme is generally used first to start the treatment process with the longer ON/OFF time Programme being used subsequently to continue the treatment process.

In the preferred embodiment, four Programmes are provided, two in each of two groups.

For many applications the facility to introduce a combination of these Programmes, in a specific sequence, within a single delivery period, and the facility to vary these combinations within a total treatment regime—dependent upon progress made—is both powerful and unique.

The currents within the Apparatus and the facility to vary both the combination of Programmes and the length of delivery time of each individual Programme within every single period of delivery, provides one unit to address all problems, which is considered unique.

The Apparatus is suitable for use on humans, and all animals, and has been designed specifically for “home use”.

### BRIEF DESCRIPTION OF THE DRAWINGS

Embodiments of the invention will now be described by way of non-limiting example with reference to the accompanying drawings, in which:

FIG. 1 is a schematic view of a micro current treatment system according to a first embodiment of the invention;

FIG. 2 is a schematic diagram of components of a pulse delivery device of the system shown in FIG. 1;

FIG. 3 is a schematic diagram of components of a programming unit of the system shown in FIG. 1;

FIG. 4 is a waveform that forms a portion of an overall waveform generated for first and second programmes and delivered by the pulse delivery device shown in FIG. 1;

FIG. 5 is a waveform that forms a portion of an overall waveform generated for third and fourth programmes and delivered by the pulse delivery device shown in FIG. 1;

FIG. 6 shows a harness that can be used as part of the system shown in FIG. 1;

FIG. 7 is a front view of a programming unit of the system shown in FIG. 1; and

FIG. 8 is a front view of a pulse delivery device of the system shown in FIG. 1.

### DETAILED DESCRIPTION OF A PREFERRED EMBODIMENT

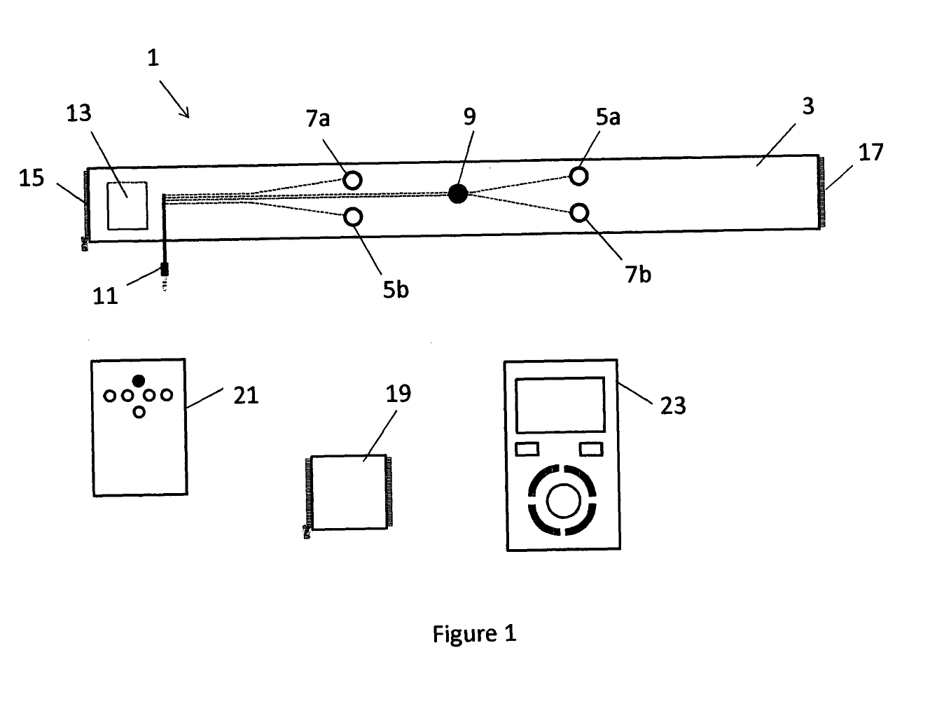

Referring to FIG. 1, there is shown a micro current system 1 according to an embodiment of the invention. The system 1 is configured for use as a therapeutic/treatment device for use by humans and animals in order to treat a wide range of conditions, ailments and injuries. As is known in the art, such systems operate by passing electrical current pulses through the wearer's tissue between an anode and cathode pair.

The system 1 comprises a flexible, belt-like web 3 made of an electrically insulating material arranged to be connected at its ends to form a loop. The web 2 is two-ply and has a first pair of electrodes 5a, 5b (anode and cathode) and a second pair of electrodes 7a, 7b provided thereon. Each pair of electrodes is electrically connected by respective wires to a plug-type connector 11 for connecting each respective electrode to a pulse delivery device 21 using the plug. The wires, indicated by dotted lines, run between the two plies of material and so are concealed and protected from wear or damage.

The electrodes pairings 5a, 5b and 7a, 7b are arranged diagonally to one another so that imaginary lines drawn between the anode and cathode of each pair intersect at a point in the region of an anchoring device 9. The anchoring device 9 retains two of the wires from electrodes 5a, 7b so that they are held firmly within the web 3 and are protected against becoming disconnected internally. In an alternative construction, all four wires can be anchored by the anchoring device 9.

This cross-wise arrangement of electrode pairings 5a, 5b, 7a, 7b is found to be particularly effective due to the current flow pattern that results across the tissue of the wearer.

A pocket or pouch 13 is provided on a face of the web 3, with an open top for receiving and holding therein the pulse delivery device 21 which can be removed and inserted as and when required and connected and disconnected to the electrodes by means of the plug 11. The plug 11 can extend within the pouch so that it may be concealed when the pulse delivery device 21 is connected and held within the pocket 13. At the narrow, lateral ends of the web 3 are connecting means so that the web 3 can form a loop-like belt to be worn around a body part of the wearer, e.g. around the waist, arm or leg. The connecting means in this case is a zip system having first and second parts 15, 17, although alternative means such as a hook and loop (e.g. Velcro) system can be used.

The web 3 can be elasticated, at least along part of its length, to allow for some resilient stretching around different sized wearers and body parts. Alternatively, or additionally, one or more extension webs 19 can be provided with zips to connect to zips 15, 17 of the main web to extend its length.

The pulse delivery device 21, to be described in detail below, is a compact unit having an output socket in the form of a female part for receiving the plug 11. The delivery device's 21 size and shape is such that it fits within the pocket 13.

The pulse delivery device 21 is configured to generate and deliver pulse sequences to the electrode pairs 5a, 5b, 7a, 7b in accordance with a sequence of programmes (Programme Sequence or PSn) with each Programme within the sequence defining a waveform to be generated as a series of waveform steps with predefined characteristics.

Programme Sequences (PSn) therefore each define a particular sequence of waveforms to be output to the electrodes and which define an overall treatment process to be applied to the wearer.

In the example to be described later on, four different Programmes are defined, i.e. Programmes 1, 2, 3 and 4. A Program Sequence (PSn) will therefore comprise a defined sequence of one or more of these programmes, e.g. 1, 3, 4, 4, 2. It has been identified by the Applicant that characteristics of the waveforms for each Programme and in particular the sequence of the Programmes are effective at treating or providing therapeutic relief of a wide range of ailments and conditions.

A remote control programming unit (hereafter “programming unit”) 23 is provided and configured to wirelessly transfer data to the pulse delivery device 21 in accordance with user inputs and commands.

In one embodiment, for example, each Programme (and therefore the waveform characteristics for each) is stored in memory of the pulse delivery device 21. This can be done as a factory setting or by means of a periodic update from a computer terminal or from the programming unit 23. In this way, at the time of use, the user simply inputs the required Programme Sequence into the programming unit 23 for transfer to the pulse delivery device 21; here, the Programme Sequence is converted into the required sequence of waveforms at the pulse delivery device 21. For example, the user may simply input 1, 3, 4, 4, 2 or a predetermined code or legend corresponding to this particular sequence, e.g. PS#1 into the programming unit 23 which is then transmitted to the pulse delivery device 21.

In an alternative embodiment, each Programme is stored in memory of the programming unit 23 so that user input of the required sequence, as described above, causes transfer of the corresponding Programmes (and their waveform characteristics) to the pulse delivery device 21 at runtime. This will of course require more data to be transmitted.

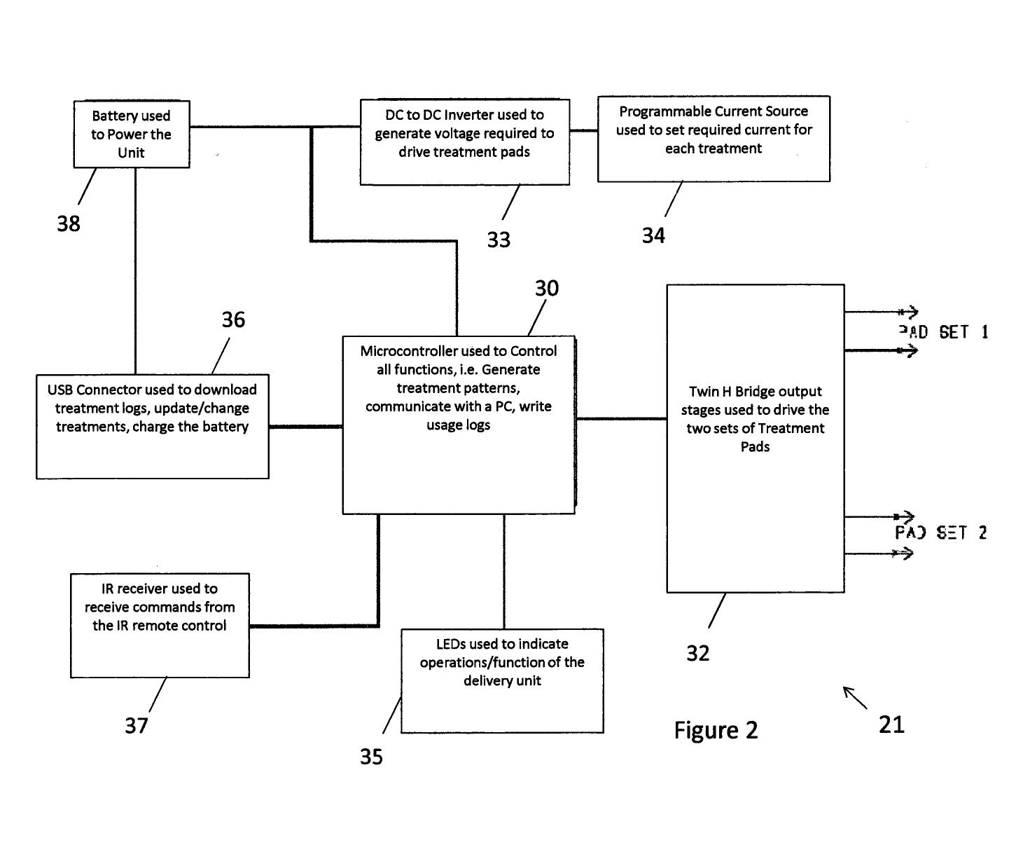

Referring to FIG. 2, there is shown a schematic diagram of components of the pulse delivery device 21. It comprises a microcontroller 30, a twin H bridge 32, a DC to DC inverter 33, a programmable current source 34, one or more LEDs 35, a USB connector 36, an infra red (IR) receiver 37, and a battery 38.

The microcontroller 30 may take any suitable form. For instance it can be a single microcontroller, plural microcontroller, a processor or plural processors.

Still referring to FIG. 2, the twin H bridge 32 is a known relay system which in this case provides the output stage for driving current to each pair of electrodes 5a, 5b and 7a, 7b in accordance with the Programmes of the Programme Sequence. The DC to DC converter 33 receives power from the battery 38 and generates the required voltage with the programmable current source 34 being used to set the required current and waveform characteristics, set using the Programme data received from the microcontroller 30. The set of one or more LEDs 35 are used to indicate different modes of operation. The USB connector 36 is used for connecting the pulse delivery device 21 to an external computer system, whether a PC, laptop, communications device or tablet computer, to enable data transfer to the memory of the delivery device and/or for charging the battery 38. The IR receiver 37 is configured to receive data and command signals from a remote control programming unit 23 to be described below.

The pulse delivery device 21 is also configured to measure and store data representing the skin impedance of the wearer between electrode pairings. This can be used for identification and calibration purposes, particularly when the system 1 is to be used in conjunction with a remote service that tailors treatments to particular users. Further details are given below.

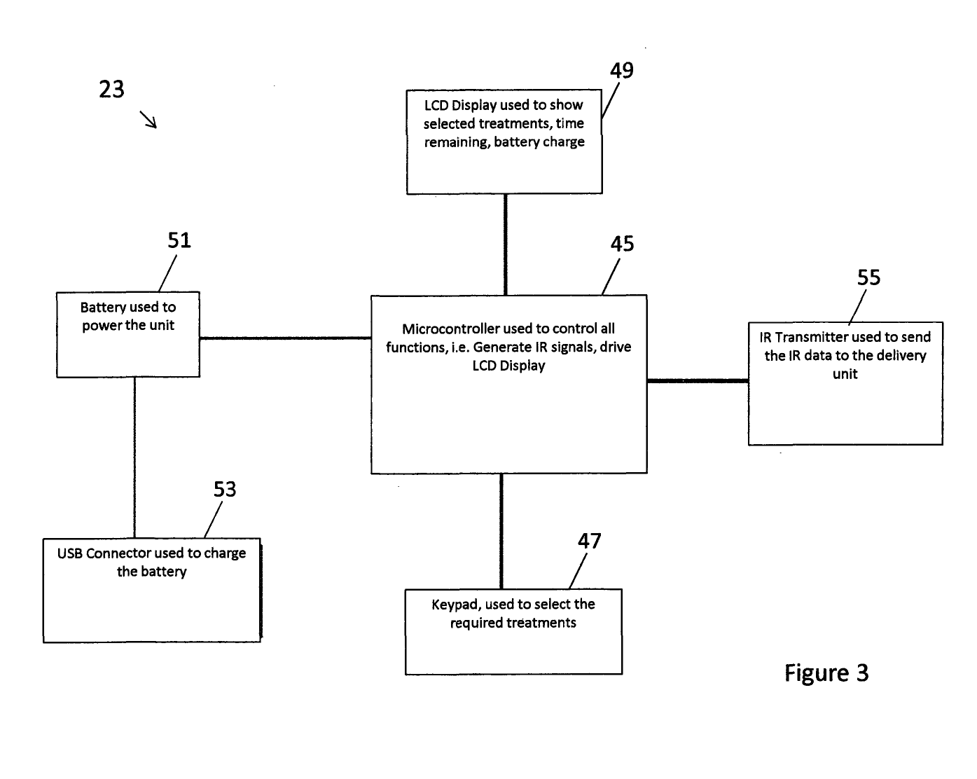

Referring to FIG. 3, there is shown a schematic diagram of components of the programming unit 23. It comprises a microcontroller 45, a keypad 47, a LCD display 49, a battery 51, a USB connector 53, and an IR transmitter 55.

The microcontroller 45 may take any suitable form. For instance it can be a single microcontroller, plural microcontroller, a processor or plural processors.

In overview, the programming unit 23 is configured to receive inputs for the uploading or transfer of data to the pulse delivery device 21 via the IR transmitter 55 communicating with the IR receiver 37 of the pulse delivery device 21. As indicated above, these inputs can comprise:
one or more Programme Sequences (PSn) for transfer to the the pulse delivery device 21;
other control commands.

Examples of Programmes and Programme Sequences (PSn) will be described below.

As indicated above, the keypad 47 can comprise hard keys, soft keys e.g. presented on a tactile touch screen display. The battery 51 is rechargeable. The USB connector 53 is used for connecting the programming unit 23 to an external computer system, whether a PC, laptop, communications device or tablet computer, to enable data transfer to the memory of the programming unit and for charging the battery 57.

Instead of using IR transmitter/receivers 55, 37 alternative wireless communications systems can be used, including WiFi and Bluetooth.

### Programmes and Programme Sequences

The pulse delivery device 21 is configured to generate electrical pulses having a predefined waveform.

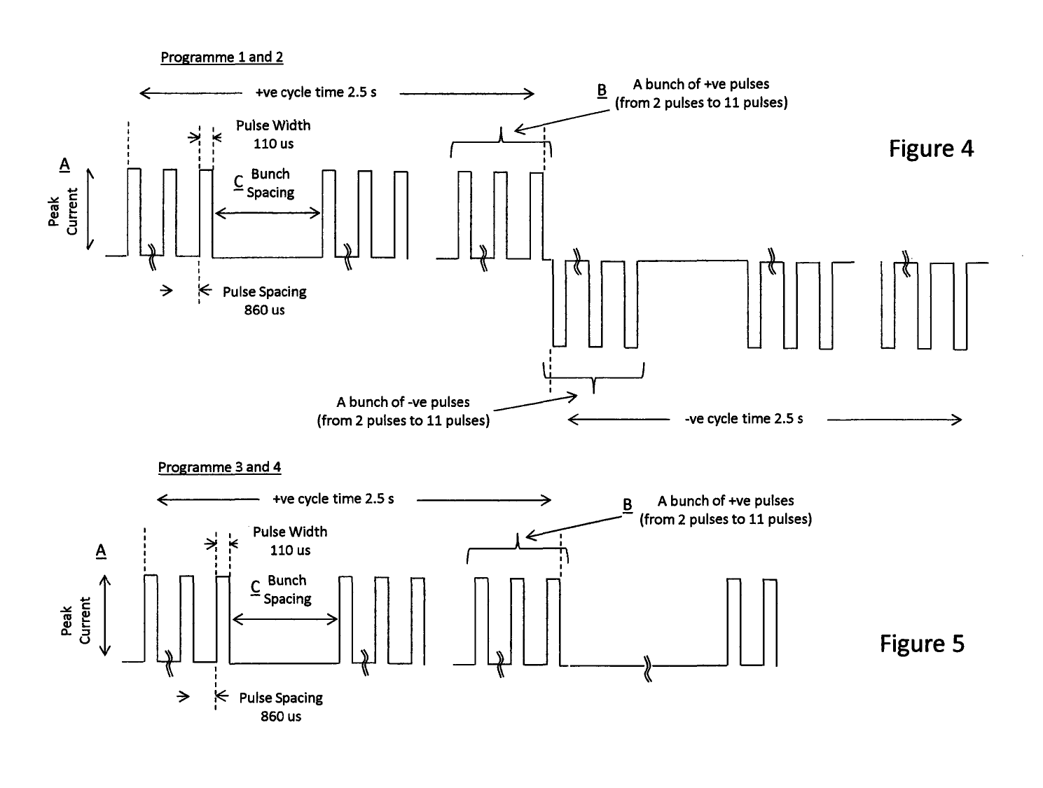

More specifically, the pulse delivery device 21 is configured to generate and output a Programme Sequence defining a plurality of Programmes selected from, in this case, four predefined Programmes (P1, P2, P3 and P4). Each Programme P1, P2, P3 and P4 comprises data defining a particular waveform in terms of a number of variables, to be discussed below, including an ON period and an OFF period, number of steps, a number of cycles within each step, a pulse peak current, a number of pulses within a bunch of pulses, and bunch spacings. FIGS. 4 and 5 indicate the meanings of peak current, pulse bunch and bunch spacing.

Annexes A-D define the waveforms corresponding to Programmes 1 to 4 respectively.

### Programme 1 (P1)

Referring to Annex A, for example, P1 comprises a waveform having the following characteristics. A five second period of the waveform is shown in FIG. 4.

First, P1 has a duration of 45 minutes.

Within this duration, a series of steps 1 to 30 are performed (and repeated as required) as shown in the table of Annex A with each step having a duration of 20 seconds made up for four cycles of the 5 second waveform shown in FIG. 4. The pulse peak current, number of pulses in a bunch of pulses and the bunch spacing for each step is generated as defined in the table.

P1 has a predefined ON time of 20 seconds and an OFF time of 10 seconds. This means that after 20 seconds of the pulsed waveform being outputted, there is an OFF (or ‘rest’) period of 10 seconds, before the programme returns to another 20 second ON time and the process continues with the next step.

So, P1 commences with step 1 (4×5 second cycles of the waveform) which happens to equal the ON time of 20 seconds. There then follows a 10 second OFF period with no pulsed output, followed by step 2 and so on. It follows that it will take 15 minutes to reach the end of step 30, and therefore 3 cycles of the 30 steps to complete the 45 minute programme duration.

### Programme 2 (P2)

Referring to Annex B, P2 comprises a waveform having the following characteristics. Again, the FIG. 4 waveform portion is referred to. P2 has a duration of 45 minutes.

In the same way as for P1, in P2 the series of steps 1 to 30 are performed (and repeated as required) as shown in the table of Annex B with each step having a duration of 20 seconds made up for four cycles of the 5 second waveform shown in FIG. 4. The pulse peak current, number of pulses in a bunch of pulses and the bunch spacing for each step is generated as defined in the table.

P2 has a predefined ON time of 10 minutes and an OFF time of 5 minutes. This means that after 10 minutes of the pulsed waveform being outputted in accordance with steps 1, 2 etc., there is an OFF (or ‘rest’) period of 5 minutes, before the programme returns to another 10 minute ON time and the process continues with the next step.

So, P2 commences with step 1 (4×5 second cycles of the waveform) and then continues to the next step and so on until step 30 is finished, at which time the 10 minute ON time finishes. The 5 minute rest period then follows, and P2 then returns to step 1 and the process repeats.

It follows that it will take 3 cycles of the 30 steps and 3 rest periods to complete the 45 minute programme duration.

### Programme 3 (P3)

Referring to Annex C, P3 comprises a waveform having the following characteristics. FIG. 5 shows a 5 second portion of the waveform.

P3 has a duration of 45 minutes.

Within this duration, a series of steps 1 to 30 are performed (and repeated as required) as shown in the table of Annex C with each step having a duration of 20 seconds made up for four cycles of the 5 second waveform shown in FIG. 5. The pulse peak current, number of pulses in a bunch of pulses and the bunch spacing for each step is generated as defined in the table.

P3 has a predefined ON time of 20 seconds and an OFF time of 10 seconds. This means that after 20 seconds of the pulsed waveform being outputted, there is an OFF (or ‘rest’) period of 10 seconds, before the programme returns to another 20 second ON time and the process continues with the next step.

So, P3 commences with step 1 (4×5 second cycles of the waveform) which happens to equal the ON time of 20 seconds. There then follows a 10 second OFF period with no pulsed output, followed by step 2 and so on. It follows that it will take 15 minutes to reach the end of step 30, and therefore 3 cycles of the 30 steps to complete the 45 minute programme duration.

### Programme 4 (P4)

Referring to Annex D, P4 comprises a waveform having the following characteristics. FIG. 5 shows a 5 second portion of the waveform. P4 has a duration of 45 minutes.

In P4 the series of steps 1 to 30 are performed (and repeated as required) as shown in the table of Annex D with each step having a duration of 20 seconds made up for four cycles of the 5 second waveform shown in FIG. 5. The pulse peak current, number of pulses in a bunch of pulses and the bunch spacing for each step is generated as defined in the table.

P4 has a predefined ON time of 10 minutes and an OFF time of 5 minutes. This means that after 10 minutes of the pulsed waveform being outputted in accordance with steps 1, 2 etc., there is an OFF (or ‘rest’) period of 5 minutes, before the programme returns to another 10 minute ON time and the process continues with the next step.

So, P4 commences with step 1 (4×5 second cycles of the waveform) and then continues to the next step and so on until step 30 is finished, at which time the 10 minute ON time finishes. The 5 minute rest period then follows, and P4 then returns to step 1 and the process repeats.

It follows that it will take 3 cycles of the 30 steps and 3 rest periods to complete the 45 minute programme duration.

The Applicant has found that providing for the aforementioned 2:1 ratio of ON and OFF times (pulsing:rest) for the programmes exhibits beneficial results.

The Applicant has also found that the particular number of cycles, pulse peak current, number of pulses in a bunch and the bunch spacing exhibits beneficial results.

The overall duration of a Programme Sequence (PSn) is set to be in the region of 6 hours maximum, which is found to exhibit beneficial results. This means a Programme Sequence can comprise up to eight Programmes.

The particular Programme Sequence (PSn) to be delivered depends on the ailment or condition that is to be treated.

The applicability of each Programme to help with particular types of condition will now be described.

The Applicant has found that Programmes 3 and 4 are useful for rapidly regenerating any tissue within a body, enabling total restoration in a fraction of normal time, with little or no scar tissue

Also, pre-existing scar tissue, both internal and external, is found to be replaced by original tissue.

Programmes 3 and 4 are also found to be successful in remedying non-union in fractures, and healing of decubitas ulcers (pressure sores, diabetic ulcers etc)

In comparison, the Applicant has found that Programmes 1 and 2 enable homeostasis for any imbalance, whether that be physical, chemical or electrical

For instance, a physical trauma will always involve swelling, bruising and inflammation and pain

Healing of the physical trauma will not be achieved, or may be seriously restricted, by the presence of swelling, bruising, inflammation and pain.

There is never a physical trauma without an associated psychological trauma. The psychological trauma has to be eliminated in order for the physical trauma to be remedied

Physical and psychological trauma both have a seriously damaging effect on the immune system, often causing/enabling many other clinical conditions.

The Applicant has found that Programmes 1 and 2 restore all of these imbalances and, in conjunction with Programmes 3 and 4, enable both psychological and physical restoration.

Therefore, an overall treatment will involve a given Programme Sequence (PSn) tailored for a particular condition.

In terms of programming the Programme Sequence (PSn) into the programming unit 23, a variety of methods might be employed. Take the example Programme Sequence of:

### PS(1): 1, 1, 2, 4, 3, 4, 4, 4

Programming might simply comprise entering these numbers into the keypad 47 of the programming unit 23. Where a Programme is repeated, then a repeat button (+) can simply be used. The notation here can be:

### PS(1): 1, +, 2, 4, 3, 4, +, +

Alternatively, different Programme Sequences (PSns) can have a unique identifier or legend, so that inputting “1” (meaning PS(1)) into the programming unit 23 is decoded by internal programming or logic to generate the above sequence for transmitting to the pulse delivery device 21.

Alternatively still, the programming unit 23 may include an optical scanner for reading a bar code or quick response (QR) code from a printed or computer-displayed menu of bar codes and QR codes, in response to which the relevant PS is identified and transmitted.

Alternatively still, the Programme Sequences (PSns) may be downloaded from a remote server, e.g. via a LAN, WAN or the Internet, in accordance with a profile or treatment programme tailored to the individual. The remote server may operate a service accessed via a web-page portal to allow users to open an account to which they can log-in and thereafter enter their details, including specifying the particular ailments/problems they wish to treat. This may be by means of a pull-down menu or a list of selectable items. In response, the server generates a suitable Programme Sequence(s) based on a predetermined rule and transmits the Programme Sequence to either the programming unit 23 or direct to the delivery device 21.

Each time the user connects their programming unit 23 or delivery device 21 to the remote server, e.g. by means of connecting the unit or device via a computer to the LAN, WAN or Internet, data can be synchronised between the two locations so that, for example, the server receives data identifying one or more of the device ID or User ID, the date of use, time of use, programme sequence(s) delivered, and programmes delivered.

In addition, data indicative of the user's skin impedance as measured over the previous period of use by the delivery device 21 may be transmitted to the server during synchronisation.

This data that is fed back to the server allows monitoring as to whether the system 1 has been used correctly, which is useful for quality control/complaint management purposes. If the user is not using the correct Programme Sequence and/or not using it for the required period, then this can be identified.

By monitoring the measured skin impedance data, it will be possible to identify whether the system 1 (and particularly the delivery device 21) has been used by a different user. It may be that a new user's skin impedance is stored on registration and subsequent values of the impedance data will clearly show if a different user has used the delivery device 21 due to a significant difference in the impedance data received. Different users may be permitted to use a single delivery device 21, in which case identification of their skin impedance can be used to calibrate and upload the relevant Programme Sequence. Alternatively, use by different people may be prohibited, in which case detection of a significantly different impedance measurement can be used to flag up inappropriate use. As well as Programme Sequences, the individual Programmes, including their waveforms and characteristics, may be downloaded from a remote server, e.g. from a LAN, WAN or the Internet.

The programming unit 23 and/or the pulse delivery device 21 and/or the Programmes may be time limited in that, at a particular date or after a particular period of use, the programming unit 23 and/or the pulse delivery device 21 is/are configured not to operate a given programme or set of programmes, at least not without some condition being met.

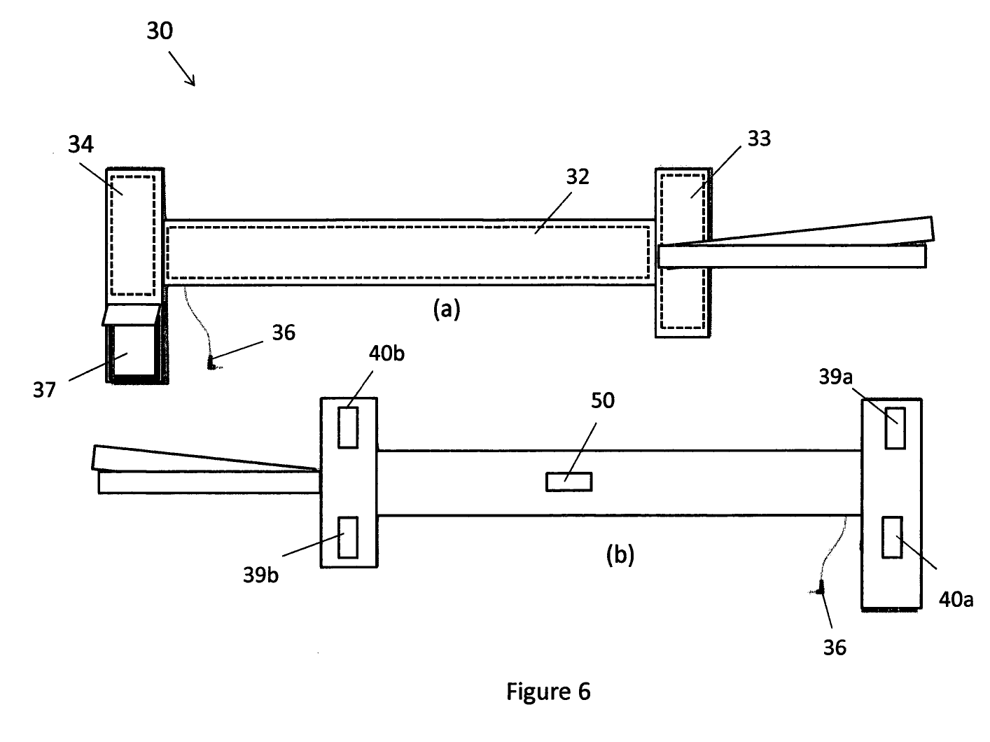

Referring now to FIG. 6, an alternative form for the web-form belt in FIG. 1 is shown. FIGS. 6(a) and 6(b) show respective sides. Here, a harness 30 is shown with a generally H or I-shaped profile, having a longitudinal central portion 32 and first and second end portions 33, 34. FIG. 6(b) shows the side of the harness 30 to be applied to the wearer's skin. There is shown a first pair of electrodes 39a, 39b and a second pair 40a, 40b. An anchor point 50 is shown in a central position and serves the same purpose as that described with reference to FIG. 1. The plug unit 36 is also shown protruding from part of the two-ply web, as is a pocket 37 for retaining and protecting the pulse delivery device 21.

The electrodes themselves, for both embodiments shown in FIGS. 1 and 6, can be formed of any metallic material, preferably an inert metal such as stainless steel. The electrodes may comprise a removable portion which in ordinary use is applied to the skin of the wearer and so can be removed for cleaning and/or washing after use. The removable portion may connect to a fixed part of the electrode by means of a press stud type mechanism.

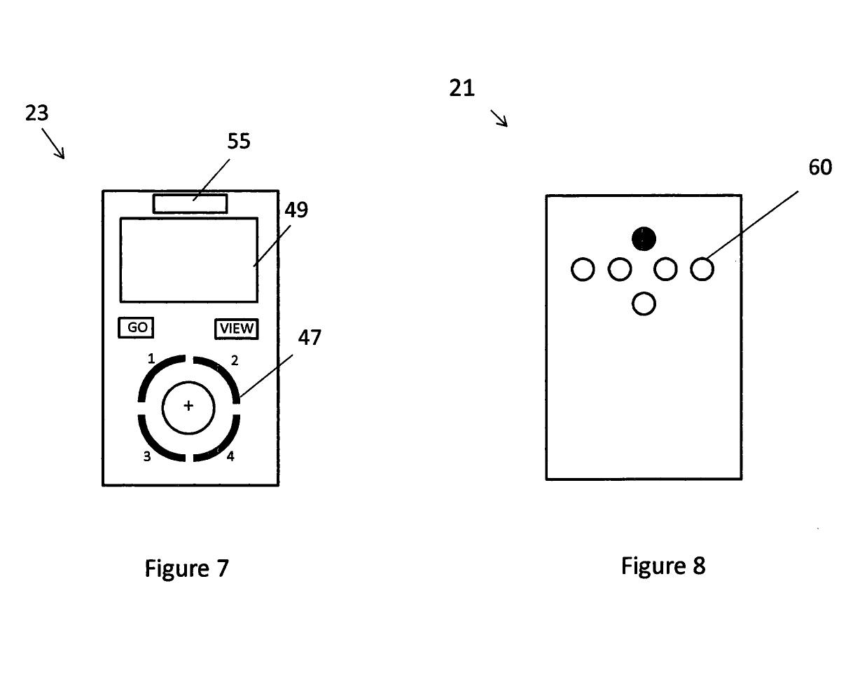

Referring to FIG. 7, a close up view of the programming unit 23 is shown with the keypad 47, LCD display 49 and IR transmitter 55 indicated. For the keypad, only four buttons are required, corresponding to each of the four programmes. The central + button permits the user to indicate a ‘repeat’ programme (as mentioned above) and/or acts as a select button. The “GO” button is operated to transmit a programme or programme sequence to the pulse delivery device 21. The “VIEW” button is used to view the current status of a Programme Sequence, i.e. what stage it is at in its overall sequence. In this respect, the LCD display 49 can display programme selections and the current status of a Programme Sequence in a number of ways, for example as a line of individual blocks or bars, each representing programmes in the Programme Sequence. The programme currently being delivered can be indicated differently from the others, e.g. by flashing or blinking. With knowledge that each programme lasts for a given amount of time, e.g. 45 minutes, then the user will at least be able to estimate the time remaining.

A more accurate figure can be achieved by displaying on the LCD display 49 a timer indicating the remaining time for the current programme.

Referring to FIG. 8, a close up view of the pulse delivery device 21 is shown. The pulse delivery device 21 comprises a number of LEDs which can be used to indicate, for example any or one of when the battery is low, when the device is ready to received data, when data is being received, when data has been received and stored, and/or which one of the four programmes is currently being delivered.

It will be appreciated that the above described embodiments are purely illustrative and are not limiting on the scope of the invention. Other variations and modifications will be apparent to persons skilled in the art upon reading the present application.

Moreover, the disclosure of the present application should be understood to include any novel features or any novel combination of features either explicitly or implicitly disclosed herein or any generalization thereof and during the prosecution of the present application or of any application derived therefrom, new claims may be formulated to cover any such features and/or combination of such features.

### ANNEX A

### Programme 1 Characteristics

ON time=20 s OFF time=10 s

| | No. of 5 | Pulse Peak | No. of pulses | Bunch |
|---|---|---|---|---|
| Step | sec cyles | current (μA) | in bunch | spacing (ms) |
| 1 | 4 | 700 | 11 | 10 |
| 2 | 4 | 650 | 10 | 9 |
| 3 | 4 | 600 | 9 | 8 |
| 4 | 4 | 550 | 8 | 7 |
| 5 | 4 | 500 | 7 | 6 |
| 6 | 4 | 450 | 6 | 5 |
| 7 | 4 | 400 | 5 | 4 |
| 8 | 4 | 350 | 4 | 3 |
| 9 | 4 | 300 | 3 | 2 |
| 10 | 4 | 250 | 2 | 1 |
| 11 | 4 | 200 | 11 | 10 |
| 12 | 4 | 150 | 10 | 9 |
| 13 | 4 | 100 | 9 | 8 |
| 14 | 4 | 50 | 8 | 7 |
| 15 | 4 | 50 | 7 | 6 |
| 16 | 4 | 50 | 6 | 5 |
| 17 | 4 | 50 | 5 | 4 |
| 18 | 4 | 100 | 4 | 3 |
| 19 | 4 | 150 | 3 | 2 |
| 20 | 4 | 200 | 2 | 1 |
| 21 | 4 | 250 | 11 | 10 |
| 22 | 4 | 300 | 10 | 9 |
| 23 | 4 | 350 | 9 | 8 |
| 24 | 4 | 400 | 8 | 7 |
| 25 | 4 | 450 | 7 | 6 |
| 26 | 4 | 500 | 6 | 5 |
| 27 | 4 | 550 | 5 | 4 |
| 28 | 4 | 600 | 4 | 3 |
| 29 | 4 | 650 | 3 | 2 |
| 30 | 4 | 700 | 2 | 1 |

### ANNEX B

### Programme 2 Characteristics

ON time=10 mins OFF time=5 mins

| | No. of 5 | Pulse Peak | No. of pulses | Bunch |
|---|---|---|---|---|
| Step | sec cyles | current (μA) | in bunch | spacing (ms) |
| 1 | 4 | 700 | 11 | 10 |
| 2 | 4 | 650 | 10 | 9 |
| 3 | 4 | 600 | 9 | 8 |
| 4 | 4 | 550 | 8 | 7 |
| 5 | 4 | 500 | 7 | 6 |
| 6 | 4 | 450 | 6 | 5 |
| 7 | 4 | 400 | 5 | 4 |
| 8 | 4 | 350 | 4 | 3 |
| 9 | 4 | 300 | 3 | 2 |
| 10 | 4 | 250 | 2 | 1 |
| 11 | 4 | 200 | 11 | 10 |
| 12 | 4 | 150 | 10 | 9 |
| 13 | 4 | 100 | 9 | 8 |
| 14 | 4 | 50 | 8 | 7 |
| 15 | 4 | 50 | 7 | 6 |
| 16 | 4 | 50 | 6 | 5 |
| 17 | 4 | 50 | 5 | 4 |
| 18 | 4 | 100 | 4 | 3 |
| 19 | 4 | 150 | 3 | 2 |
| 20 | 4 | 200 | 2 | 1 |
| 21 | 4 | 250 | 11 | 10 |
| 22 | 4 | 300 | 10 | 9 |
| 23 | 4 | 350 | 9 | 8 |
| 24 | 4 | 400 | 8 | 7 |
| 25 | 4 | 450 | 7 | 6 |
| 26 | 4 | 500 | 6 | 5 |
| 27 | 4 | 550 | 5 | 4 |
| 28 | 4 | 600 | 4 | 3 |
| 29 | 4 | 650 | 3 | 2 |
| 30 | 4 | 700 | 2 | 1 |

### ANNEX C

### Programme 3 Characteristics

ON time=20 s OFF time=10 s

| | No. of 5 | Pulse Peak | No. of pulses | Bunch |
|---|---|---|---|---|
| Step | sec cyles | current (μA) | in bunch | spacing (ms) |
| 1 | 4 | 200 | 11 | 10 |
| 2 | 4 | 150 | 10 | 9 |
| 3 | 4 | 150 | 9 | 8 |
| 4 | 4 | 200 | 8 | 7 |
| 5 | 4 | 200 | 7 | 6 |
| 6 | 4 | 250 | 6 | 5 |
| 7 | 4 | 250 | 5 | 4 |
| 8 | 4 | 200 | 4 | 3 |
| 9 | 4 | 200 | 3 | 2 |
| 10 | 4 | 150 | 2 | 1 |
| 11 | 4 | 150 | 11 | 10 |
| 12 | 4 | 200 | 10 | 9 |
| 13 | 4 | 200 | 9 | 8 |
| 14 | 4 | 250 | 8 | 7 |
| 15 | 4 | 250 | 7 | 6 |
| 16 | 4 | 200 | 6 | 5 |
| 17 | 4 | 200 | 5 | 4 |
| 18 | 4 | 150 | 4 | 3 |
| 19 | 4 | 150 | 3 | 2 |
| 20 | 4 | 200 | 2 | 1 |

### ANNEX D

### Programme 4 Characteristics

ON time=10 mins OFF time=5 mins

| | No. of 5 | Pulse Peak | No. of pulses | Bunch |
|---|---|---|---|---|
| Step | sec cyles | current (μA) | in bunch | spacing (ns) |
| 1 | 4 | 200 | 11 | 10 |
| 2 | 4 | 150 | 10 | 9 |
| 3 | 4 | 150 | 9 | 8 |
| 4 | 4 | 200 | 8 | 7 |
| 5 | 4 | 200 | 7 | 6 |
| 6 | 4 | 250 | 6 | 5 |
| 7 | 4 | 250 | 5 | 4 |
| 8 | 4 | 200 | 4 | 3 |
| 9 | 4 | 200 | 3 | 2 |
| 10 | 4 | 150 | 2 | 1 |
| 11 | 4 | 150 | 11 | 10 |
| 12 | 4 | 200 | 10 | 9 |
| 13 | 4 | 200 | 9 | 8 |
| 14 | 4 | 250 | 8 | 7 |
| 15 | 4 | 250 | 7 | 6 |
| 16 | 4 | 200 | 6 | 5 |
| 17 | 4 | 200 | 5 | 4 |
| 18 | 4 | 150 | 4 | 3 |
| 19 | 4 | 150 | 3 | 2 |
| 20 | 4 | 200 | 2 | 1 |

1. Apparatus for treatment of an animal or human comprising:
a pulse delivery system for generating a series of electrical current pulses having a pre-programmed waveform; and
a flexible belt or belt-like carrier arranged in use to be secured to, on or around an animal or human and comprising a pair of spaced-apart electrodes for making contact with the skin of the animal or human, and a connector for electrically connecting an output terminal of the pulse delivery system to the electrodes so that in use the current pulses are delivered between the electrodes across or through underlying tissue of the animal or human.

2. Apparatus according to claim 1, wherein the carrier includes a holding means for detachably holding the pulse delivery system thereon.

3. Apparatus according to claim 2, wherein the holding means comprises a pocket or pouch.

4. Apparatus according to any preceding claim, wherein the connector comprises a plug/socket electrically connected to the pair of electrodes and which is configured to be detachably connected to a corresponding socket/plug of the pulse delivery system.

5. Apparatus according to claim 4 when dependent on claim 3, wherein the plug/socket is connected to the electrodes by means of a lead which extends from the electrodes to within the pocket or pouch.

6. Apparatus according to any preceding claim, wherein two pairs of electrodes are provided, each pair comprising one anode and one cathode.

7. Apparatus according to claim 6, wherein the electrodes of the pairs are arranged such that the imaginary line between the anode and cathode of each pair intersect one another.

8. Apparatus according to any preceding claim, wherein each electrode comprises a first part fixed to the carrier and a detachable part which, in use, makes contact with the skin or the animal or human.

9. Apparatus according to claim 8, wherein the detachable part is connected to the fixed part by means of a press-stud mechanism.

10. Apparatus according to any preceding claim, wherein the carrier comprises a flexible web of material having first and second end regions which are connectable to one another to form a looped belt or harness by means of a zip.

11. Apparatus according to claim 10, further comprising one or more extension parts attachable to the end regions by means of a zip.

12. Apparatus according to any preceding claim, wherein the carrier comprises a generally I or H-shaped flexible web of material.

13. Apparatus according to any preceding claim, wherein the pulse delivery system comprises means for storing a plurality of programmes, each programme defining characteristics of a respective waveform, and means for executing the programmes to generate a waveform or sequence of waveforms using one or more of the programmes.

14. Apparatus according to claim 13, wherein each programme defines a waveform in terms of an ON time in which the waveform comprises a sequence of pulses and an OFF time in which the waveform comprises no pulses.

15. Apparatus according to claim 14, wherein the ratio of ON:OFF times is substantially 2:1.

16. Apparatus according to any one of claims 13 to 15, wherein each programme defines a sequence of steps of predetermined duration, each step defining a different characteristic in terms of one or more of peak pulse current, number of pulse bunches, and/or pulse spacing than that of a previous step.

17. Apparatus according to any one of claims 13 to 16, wherein each programme is configured to run for substantially 45 minutes before ending.

18. Apparatus according to any one of claims 13 to 17, wherein the pulse delivery system comprises means for storing a programme sequence defining the number and order of programmes to be generated one after the other over a predefined time period.

19. Apparatus according to any one of claims 13 to 18, further comprising means for receiving data representing the plurality of programmes and/or a programme sequence from a remote device.

20. Apparatus according to claim 19, further comprising a remote programming unit including an input means into which programmes and/or a programme sequence can be entered.

21. Apparatus according to claim 20, further comprising means for receiving a programme sequence by way of receiving an identifier or code which is converted into the programme sequence by decoding logic or a decoding program on the pulse delivery system.

22. Apparatus according to any one of claims 13 to 21, wherein the pulse delivery system or remote programming unit comprises a display and means configured to output to said display an indication of the time remaining to generate pulses for a programme or programme sequence.

23. Apparatus according to any one of claims 13 to 22, wherein the or each programme is time limited such that after a predetermined time from or by a predetermined date, the pulse delivery system will no longer generate pulses using the or each programme.

24. Apparatus according to any preceding claim, further comprising means to connect to a remote server and transmit data indicative of use of the pulse delivery system, including one or more of user identity, time/date of ues, and programme(s) delivered.

25. Apparatus according to any preceding claim, wherein the pulse delivery system further comprises means to measure and record the skin impedance of a wearer.

26. Apparatus according to claim 25 when dependent on claim 24, further comprising means to transmit the skin impedance measurement to the remote server.

27. Apparatus for treatment of an animal or human comprising a pulse delivery system for generating a series of electrical current pulses having a pre-programmed waveform, the pulse delivery system comprising means for storing a plurality of programmes, each programme defining characteristics of a respective waveform, and means for executing the programmes to generate a waveform or sequence of waveforms using one or more of the programmes for output to electrodes.

28. Apparatus according to claim 27, further comprising a remote control unit configured to receive inputs defining one or more programmes or sequence of programmes and for transferring said programmes or sequence of programmes to the pulse delivery system over a wireless link.
